(19) United States
(12) Patent Application Publication
(10) Pub. No.: US 2023/0143850 A1
(43) Pub. Date: May 11, 2023

(54) PROTECTIVE SOLUTION FOR ISOLATING MITOCHONDRIA, USE THEREOF, KIT COMPRISING THE SAME, AND METHOD FOR ISOLATING MITOCHONDRIA

(71) Applicant: TAIWAN MITOCHONDRION APPLIED TECHNOLOGY CO., LTD., Zhubei City (TW)

(72) Inventors: Han-Chung CHENG, Zhubei City (TW); Chih-Kai HSU, Zhubei City (TW)

(73) Assignee: TAIWAN MITOCHONDRION APPLIED TECHNOLOGY CO., LTD., Zhubei City (TW)

(21) Appl. No.: 18/093,767

(22) Filed: Jan. 5, 2023

Related U.S. Application Data
(63) Continuation-in-part of application No. PCT/MY2021/050056, filed on Jul. 7, 2021.

(30) Foreign Application Priority Data
Jul. 8, 2020 (TW) 109123093

(51) Int. Cl. C12M 1/26; C12N 5/07

(52) U.S. Cl. CPC C12M 33/04; C12N 5/06

(57) ABSTRACT

The embodiments according to the present disclosure provide a protective solution, a kit, and a method for isolating mitochondria. The kit includes an extraction tube, a protective solution, and a suction needle. The extraction tube is used to contain cells. The protective solution is used to mix with the cells in the extraction tube to form a mixture solution, and the osmolarity of the protective solution is greater than 0 and less than or equal to 220 mOsm/L. The suction needle is used to suck the mixed solution back and forth to facilitate the release of mitochondria from cells. This method and the protective solution may isolate mitochondria from cells with high efficiency in a simple and convenient way, and the isolated mitochondria have excellent good function.

### CROSS-REFERENCE TO RELATED APPLICATIONS

This application is a continuation-in-part of International Application No. PCT/MY2021/050056, filed on Jul. 7, 2021, which claiming priority to Patent Application No(s). 100123093 filed in Taiwan (R.O.C.) on Jul. 8, 2020, the entire contents of which are hereby incorporated by reference.

### BACKGROUND

### 1. Technical Field

This disclosure relates to a protective solution for isolating mitochondria, use thereof, a kit comprising the same, and a method for isolating mitochondria.

### 2. Related Art

A mitochondrion is one of the important organelles in a cell. Mitochondria not only supply energy (adenosine triphosphate, ATP) for cells but also involve the regulation of oxidative stress, apoptosis, communication between cells, and transportation of signals. Recently, many studies have reported that aging and the formation of diseases have a close relationship with mitochondria (Braticet al., 2013). Some studies have further shown that a technique of mitochondrial transplantation can repair the damages to cells and tissues (Pacak et al., 2015; Cowanet al., 2017).

Therefore, how to obtain mitochondria and maintain their function and activity in an efficient way is one of the aims of development.

### SUMMARY

Accordingly, this disclosure provides a protective solution for isolating mitochondria, a kit comprising the same, and a method for isolating mitochondria. In this disclosure, mitochondria may be isolated from cells with high efficiency in a simple and convenient way, and the isolated mitochondria may maintain their function and activity.

According to one embodiment of the present disclosure, a kit for isolating mitochondria comprises an extraction tube for containing cells; a protective solution for forming a mixed solution with the cells in the extraction tube; and a sucking needle for sucking the mixed solution back and forth.

According to one embodiment of the present disclosure, a method for isolating mitochondria comprises mixing cells and a protective solution to form a mixed solution, with a osmolarity of the protective solution greater than 0 and less than or equal to 220 mOsm/L; rubbing the cells in the mixed solution to damage cell membranes of the cells to facilitate an entry of the protective solution into the cells and destroy the cell membranes of the cells;

centrifuging the mixed solution; and collecting supernatant comprising mitochondria obtained after centrifuging.

According to one embodiment of the present disclosure, a protective solution for isolating and protecting mitochondria from cells is provided, wherein a osmolarity of the protective solution is greater than 0 and less than or equal to 220 mOsm/L.

According to one embodiment of the present disclosure, a use of a protective solution in isolating mitochondria from cells and maintaining the activity of mitochondria is provided, wherein the protective solution is a hypotonic solution and comprises sodium chloride, glucose, sodium dihydrogen phosphate, or mannitol.

In view of the above description, the present disclosure provides a protective solution for isolating mitochondria, a kit comprising the same, and a method for isolating mitochondria. The cell membranes of the cells may be destroyed by the sucking needle and the protective solution in the kit through the friction of the cells in the sucking needle with the help of the protective solution, and this way of destroying the cell membranes may not damage the mitochondria in the cells. In addition, the osmolarity of the protective solution is greater than 0 and less than or equal to 220 mOsm/L so that the cell membranes of the cells may be destroyed with the help of the hypotonicity of the protective solution to release the mitochondria. Therefore, the mitochondria may be isolated from cells with high efficiency in a simple and convenient way, and the isolated mitochondria may have excellent function and activity.

### BRIEF DESCRIPTION OF THE DRAWINGS

The present disclosure will become more fully understood from the detailed description given hereinbelow and the accompanying drawings which are given by way of illustration only and thus are not limitative of the present disclosure and wherein:

FIG. 1 is a schematic view illustrating a kit for isolating mitochondria according to the first embodiment of the present disclosure.

FIG. 2 is a flow chart illustrating a method for isolating mitochondria by using the kit according to the first embodiment of the present disclosure.

FIG. 3 is a schematic view illustrating the implementation of the kit according to the first embodiment of the present disclosure.

FIG. 4 is a schematic view illustrating a kit for isolating mitochondria according to the second embodiment of the present disclosure.

FIG. 5 is a flow chart illustrating a method for isolating mitochondria by using the kit according to the second embodiment of the present disclosure.

FIG. 6 shows the extraction efficiency for different numbers of times for sucking back and forth and different lengths of the needle.

FIG. 7 shows the extraction efficiency for different numbers of the cells and different volumes of the protective solution.

FIG. 8 shows the extraction efficiency for different times for standing.

FIG. 9 shows the function of mitochondria isolated from the peripheral blood mononuclear cells by the protective solution comprising NaCl with different osmolarities.

FIG. 10 shows the extraction efficiency of mitochondria isolated from the peripheral blood mononuclear cells by the protective solution comprising NaCl with different osmolarities.

FIG. 11 shows the function of mitochondria isolated from the adipose-derived stem cells by the protective solution comprising NaCl, glucose, NaH₂PO₄, or mannitol with different osmolarities.

FIG. 12 shows the extraction efficiency of mitochondria isolated from the adipose-derived stem cells by the protective solution comprising NaCl, glucose, NaH₂PO₄, or mannitol with different osmolarities.

FIG. 13 shows the function of mitochondria isolated from the adipose-derived stem cells by the protective solution comprising different compositions with an osmolarity of 42.8 mOsm/L.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawings.

The embodiments according to the present disclosure provide a kit, a method, and a protective solution for isolating mitochondria from cells and protecting the isolated mitochondria. The aforementioned cells may include any cells having mitochondria, for example, peripheral blood mononuclear cells, hematopoietic stem cells, embryonic stem cells, mesenchymal stem cells, adipose-derived mesenchymal stem cells, umbilical cord mesenchymal stem cells, CD34+ stem cells, amniotic fluid mesenchymal stem cells, bone marrow mesenchymal stem cells, induced pluripotent stem cells, neural stem cells, epithelial stem cells, skin stem cells, somatic stem cells, somatic cells, smooth muscle cells, liver cells, kidney cells, cardiomyocytes, monocytes, lymphocytes, keratinocytes, and hair follicle cells.

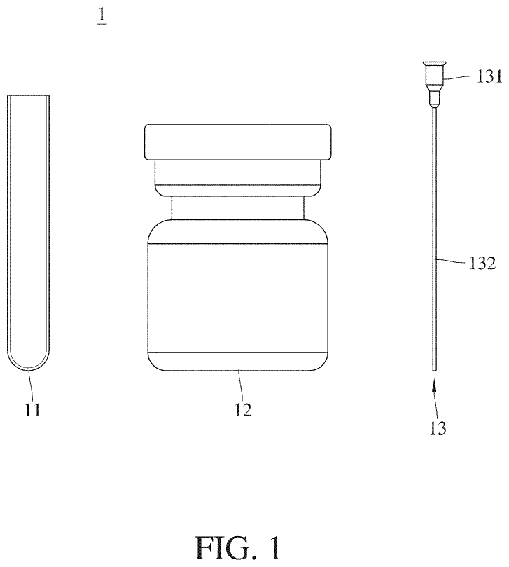

A kit for isolating mitochondria according to the first embodiment of the present disclosure will be described below. Please refer to FIG. 1, wherein FIG. 1 is a schematic view illustrating a kit for isolating mitochondria according to the first embodiment of the present disclosure. The kit 1 for isolating mitochondria in the first embodiment of the present disclosure comprises an extraction tube 11, a protective solution 12, and a sucking needle 13.

The extraction tube is usually a cylinder. The extraction tube 11 has a closed bottom and an opening. The extraction tube is used to contain cells and solutions. The cells are cells that have mitochondria. In this embodiment, the extraction tube 11 is a cylinder tube, but the present disclosure is not limited thereto. In other embodiments, the extraction tube may be a tube in any shape as long as acceptable to the centrifuge.

The protective solution 12 is used for mixing with the cells in the extraction tube 11 to form a mixed solution. In the first embodiment, the osmolarity of the protective solution 12 is greater than 0 and less than or equal to 220 mOsm/L, but the present disclosure is not limited thereto. In other embodiments, the protective solution may be any common buffer that can preserve organelles or maintain the activity of organelles. In the first embodiment, the protective solution 12 is contained in a container different from the extraction tube 11, but the present disclosure is not limited thereto. In other embodiments, the protective solution may be contained in the extraction tube so that the cells may be directly added to the extraction tube and mixed with the protective solution contained in the extraction tube to form the mixed solution.

The sucking needle 13 has a connection end 131 and a tip end 132. The connection end 131 is used to connect to a syringe. The tip end 132 is used to suck or inject solutions. The sucking needle 13 is used to suck back and forth the mixed solution comprising the cells and the protective solution 12 in the extraction tube 11, and the cells in the mixed solution may be rubbed back and forth with the inner wall of the needle in the sucking needle 13. The cell membranes are damaged due to the friction thereby releasing the mitochondria. In the first embodiment, the length of the sucking needle 13 corresponds to the length of the extraction tube 11 to be 70 mm, but the present disclosure is not limited thereto. In other embodiments, the length of the sucking needle may be 15 mm. In the first embodiment, the inner diameter of the sucking needle 13 is 0.337 mm, but the present disclosure is not limited thereto. In other embodiments, the inner diameter of the sucking needle may be 0.318 to 0.356 mm.

In the first embodiment, the extraction tube 11, the protective solution 12, and the sucking needle 13 are sterile, but the present disclosure is not limited thereto. In other embodiments, they may be sterilized before used.

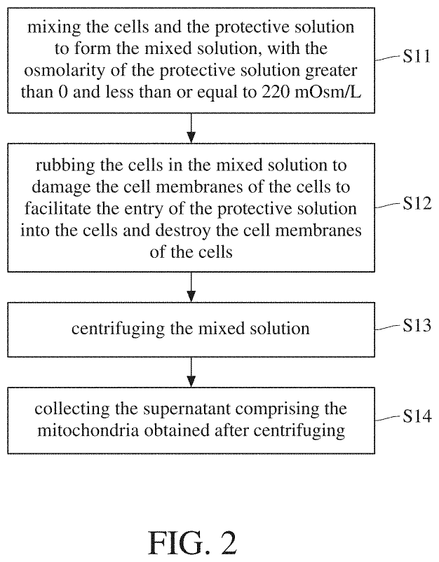

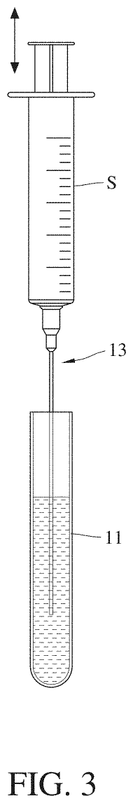

A method for isolating mitochondria by using the kit according to the first embodiment of the present disclosure will be described below. Please refer to FIGS. 2 and 3, wherein FIG. 2 is a flow chart illustrating a method for isolating mitochondria by using the kit according to the first embodiment of the present disclosure, and FIG. 3 is a schematic view illustrating the implementation of the kit according to the first embodiment of the present disclosure.

Firstly, cells and a protective solution 12 are mixed in an extraction tube 11 to form a mixed solution, and the osmolarity of the protective solution is greater than 0 and less than or equal to 220 mOsm/L (S11). In detail, the cells and the protective solution 12 may be added to the extraction tube 11 by using other needles or pipettes or by pouring, to form the mixed solution. The sequence of adding the cells and adding the protective solution 12 is not limited. The cells may be any cell having mitochondria, such as peripheral blood mononuclear cells, platelets, somatic stem cells, adipose-derived stem cells, embryonic stem cells, mesenchymal stem cells, hematopoietic stem cells, amniotic stem cells, amniotic fluid stem cells, neural stem cells, hair follicle stem cells, olfactory ensheathing stem cells, CD34+ stem cells, bone marrow stem cells, skeletal muscle cells, hepatic cells, kidney cells, fibroblasts, endothelial cells, oral epithelial cells, myocardial cells, nerve cells, keratinocytes, epithelial cells, etc. The peripheral blood mononuclear cells or platelets may be obtained by stratifying the peripheral blood and collecting the desired mononuclear cells or platelets by any known method. In this embodiment, the protective solution 12 is a hypotonic solution, the osomolarity is greater than 0 and less than or equal to 220 mOsm/L, and each milliliter of the protective solution 12 may be used for treating 1×10⁶ to 5×10⁶ cells, but the present disclosure is not limited thereto. In this embodiment, the cells and the protective solution 12 are mixed in the extraction tube 11, but the present disclosure is not limited thereto. In other embodiments, the cells and the protective solution 12 may be mixed in a container other than the extraction tube and then added to the extraction tube.

Subsequently, the cells in the mixed solution are rubbed to damage the cell membranes of the cells to facilitate the entry of the protective solution 12 into the cells and destroy the cell membranes of the cells (S12). In detail, the mixed solution in the extraction tube 11 is sucked back and forth by using a sucking needle 13 and a syringe S. As shown in FIG. 3, the mixed solution in the extraction tube 11 is sucked back and forth several times (e.g., 5 times) by using the sucking needle 13 and the syringe S. Thereby, the cells in the mixed solution are rubbed back and forth with the inner wall of the needle in the sucking needle 13. The cell membranes are damaged due to the friction so that the protective solution 12 enters into the cells through the damaged membranes to destroy the cell membrane, thereby releasing the mitochondria.

Subsequently, the mixed solution is centrifuged (S13). In detail, the mixed solution after being sucked back and forth by the sucking needle 13 is centrifuged at 1500 to 2500 rpm for 5 to 15 minutes. The pellet comprising cell debris and the supernatant comprising the mitochondria are stratified by the centrifugation.

Finally, the supernatant comprising the mitochondria obtained after the centrifugation is collected (S14). In detail, the supernatant comprising the mitochondria may be collected by using other needles or pipettes or by pouring so as to achieve the purpose of isolating the mitochondria.

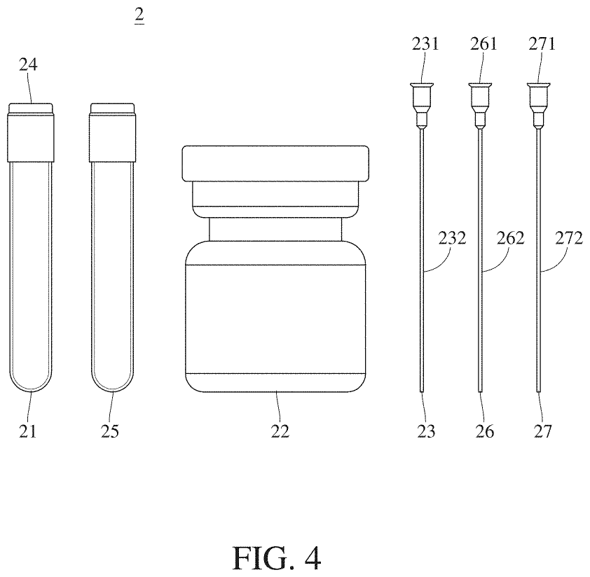

A kit for isolating mitochondria according to the second embodiment of the present disclosure will be described below. Please refer to FIG. 4, wherein FIG. 4 is a schematic view illustrating a kit for isolating mitochondria according to the second embodiment of the present disclosure. The second embodiment is similar to the first embodiment, and only differences will be described below. In addition to an extraction tube 21, a protective solution 22, and a sucking needle 23, the kit 2 in the second embodiment of the present disclosure further comprises a stopper 24, a balance tube 25, a cell collection needle 26, and a mitochondria collection needle 27. The extraction tube 21, the protective solution 22, and the sucking needle 23 in the second embodiment are the same as the extraction tube 11, the protective solution 12, and the sucking needle 13 in the first embodiment. Therefore, please refer to the description of the first embodiment for the details of these components, and the details are not repeated here.

The size of the stopper 24 corresponds to the size of the opening of the extraction tube 21. The stopper 24 is used to seal the extraction tube 21 to prevent the solution in the extraction tube 21 from being contaminated by the outside. In this embodiment, the stopper 24 is a rubber stopper, and the rubber stopper maintains the seal of the extraction tube 21 after being pierced by the needle, but the present disclosure is not limited thereto. In other embodiments, the extraction tube may be closed by a cap or a lid rather than a rubber stopper.

The weight of the balance tube 25 is equal to that of the extraction tube 21. The balance tube 25 is used to keep balance with the extraction tube 21 during centrifugation. In detail, when the extraction tube 21 is sealed by the stopper 24, the balance tube 25 may have a stopper, a cap, or a lid that has the same weight as the stopper 24 to keep balance during centrifugation. When the extraction tube 21 contains a solution, the balance tube 25 may be added with the same weight of the solution to keep balance during centrifugation. In other embodiments, the balance tube may be the same as the extraction tube, or there may be not a balance tube as long as the turntable of the centrifuge keeps balance during centrifugation.

The cell collection needle 26 has a connection end 261 and a tip end 262. The connection end 261 is used to connect to a syringe. The tip end 262 is used to suck or inject solutions. The cell collection needle 26 is used to suck a solution comprising cells and inject the solution into the extraction tube 21. The length and the inner diameter of the cell collection needle 26 may be the same as or different from those of the sucking needle 23. In other embodiments, there may be not a cell collection needle, and the solution comprising the cells may be injected into the extraction tube by a pipette.

The mitochondria collection needle 27 has a connection end 271 and a tip end 272. The connection end 271 is used to connect to a syringe. The tip end 272 is used to suck or inject solutions. The mitochondria collection needle 27 is used to collect the supernatant comprising mitochondria. The length and the inner diameter of the mitochondria collection needle 27 may be the same as or different from those of the sucking needle 23. In other embodiments, there may be not a mitochondria collection needle, and the supernatant comprising the mitochondria may be collected by a pipette.

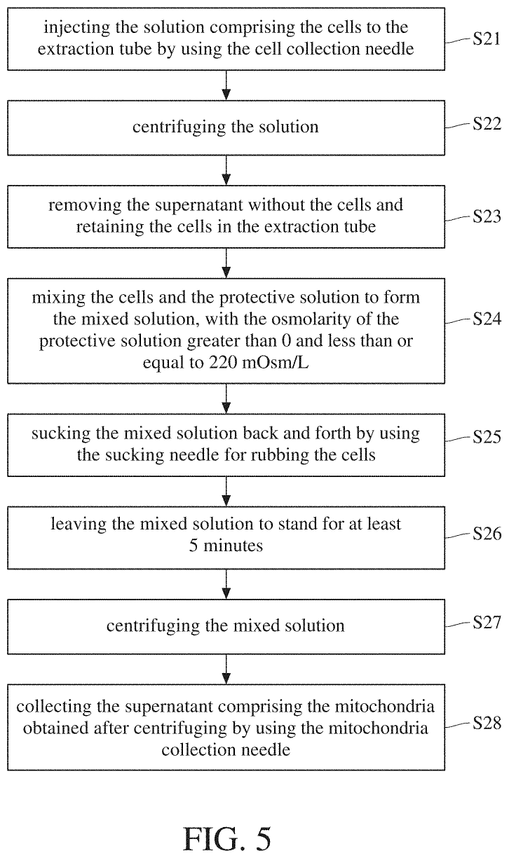

A method for isolating mitochondria by using the kit according to the second embodiment of the present disclosure will be described below. Please refer to FIG. 5, wherein FIG. 5 is a flow chart illustrating a method for isolating mitochondria by using the kit according to the second embodiment of the present disclosure.

Firstly, a solution comprising cells is injected to an extraction tube 21 by using a cell collection needle 26 (S21). In detail, the solution comprising the cells is injected into the extraction tube 21 sealed with a stopper 24 by using the cell collection needle 26. The cells may be any cell having mitochondria, such as peripheral blood mononuclear cells, platelets, somatic stem cells, adipose-derived stem cells, embryonic stem cells, mesenchymal stem cells, hematopoietic stem cells, amniotic stem cells, amniotic fluid stem cells, neural stem cells, hair follicle stem cells, olfactory ensheathing stem cells, CD34+ stem cells, bone marrow stem cells, skeletal muscle cells, hepatic cells, kidney cells, fibroblasts, endothelial cells, oral epithelial cells, myocardial cells, nerve cells, keratinocytes, epithelial cells, etc. The peripheral blood mononuclear cells or platelets may be obtained by stratifying the peripheral blood by any known method and collecting the desired mononuclear cells or platelets by using the cell collection needle 26. Here, the solution comprising the cells is injected by the cell collection needle 26 rather than the sucking needle 23, which may prevent the protective solution 22 contacted with the sucking needle 23 from being contaminated.

Subsequently, the solution is centrifuged (S22). In detail, the balance tube 25 is added with liquid to have the same weight as the extraction tube 21 which contains the solution of the cells. The extraction tube 21 containing the solution of the cells and the balance tube 25 are centrifuged at 1000 to 1500 rpm for 5 to 10 minutes so that the solution of the cells is stratified to form the pellet comprising the cells and the supernatant without cells.

Subsequently, the supernatant without the cells after centrifugation is removed, and the cells are retained in the extraction tube 21 (S23). In detail, the supernatant without the cells is removed by using the cell collection needle 26, and the cells are retained in the extraction tube 21. Removing the supernatant without the cells may increase the concentration of the cells in the extraction tube 21 and prevent the protective solution 22 subsequently added from being diluted by other liquid which decreases the extraction efficiency. Here, the supernatant is removed by the cell collection needle 26 rather than the sucking needle 23, which may prevent the protective solution 22 contacted with the sucking needle 23 from being contaminated.

Subsequently, the cells and the protective solution 22 are mixed in the extraction tube 21 to form a mixed solution, and the osmolarity of the protective solution 22 is greater than 0 and less than or equal to 220 mOsm/L (S24). In detail, a proper amount of the protective solution 22 is added to the extraction tube 21 containing the cells by the sucking needle 23, to form the mixed solution. In this embodiment, the protective solution 22 is a hypotonic solution, and the osmolarity is greater than 0 and less than or equal to 220 mOsm/L, but the present disclosure is not limited thereto. In other embodiments, the protective solution may be any common buffer that can preserve organelles or maintain the activity of organelles. In the second embodiment, each milliliter of the protective solution 22 may be used for treating approximately 1×10⁶ to 5×10⁶ cells.

Subsequently, the mixed solution in the extraction tube 21 is sucked back and forth by the sucking needle 23 and a syringe S (S25). In detail, as shown in FIG. 3, the mixed solution in the extraction tube 21 is sucked back and forth several times (e.g., 5 times) by using the sucking needle 23 and the syringe S. Thereby, the cells in the mixed solution are rubbed back and forth with the inner wall of the needle in the sucking needle 23. The cell membranes are damaged due to the friction so that the protective solution 22 enters into the cells through the damaged membranes to destroy the cell membrane, thereby releasing the mitochondria.

Subsequently, the mixed solution stands for at least five minutes (S26). In detail, the mixed solution is placed at a stable place, and the time for standing is at least five minutes, but the present disclosure is not limited thereto. Standing of the mixed solution allows the hypotonic protective solution has enough time to diffuse to facilitate the destruction of the damaged cell, thereby facilitating the release of the mitochondria.

Subsequently, the mixed solution is centrifuged (S27). In detail, the balance tube 25 is added with liquid to have the same weight as the extraction tube 21 which contains the mixed solution. The extraction tube 21 containing the mixed solution after standing and the balance tube 25 are centrifuged at 1500 to 2500 rpm for 5 to 15 minutes. The pellet comprising cell debris and the supernatant comprising the mitochondria are stratified by the centrifugation.

Finally, the supernatant comprising the mitochondria obtained after the centrifugation is collected by using a mitochondria collection needle 27 (S28). In detail, the supernatant comprising the mitochondria is collected by the mitochondria collection needle 27, thereby isolating the mitochondria from the cell debris in the mixed solution. Here, the supernatant comprising the mitochondria is collected by the mitochondria collection needle 27 rather than the sucking needle 23, which may prevent the supernatant comprising the mitochondria from being contaminated by the sucking needle 23 contacted with the protective solution and the mixed solution.

Experiments 1 to 5 demonstrate isolating mitochondria by the method using the kit according to the second embodiment of the present disclosure, and the extraction efficiency and the function of the isolated mitochondria will be shown.

Specifically, the peripheral blood mononuclear cells or the adipose-derived stem cells are collected by using the cell collection needle, and the cells are injected into the extraction tube. The peripheral blood mononuclear cells are obtained by drawing 8 to 20 mL of the peripheral blood from veins, centrifuging the blood at 2000 rpm for 10 min to stratify it, and collecting the layer of the peripheral blood mononuclear cells. The extraction tube containing the cells is centrifuged at 1000 rpm for 5 min to precipitate the cells, and the supernatant is removed by the cell collection needle. Then, 1 to 2 mL of the protective solution is added to the extraction tube containing the cells to form the mixed solution. The mixed solution is sucked back and forth several times by the sucking needle. Then, the mixed solution stands for at least 5 min and then centrifuged at 2000 rpm for 10 min, and the mixed solution is stratified to form the supernatant comprising mitochondria and the pellet comprising the cell debris. The supernatant comprising the mitochondria after centrifugation is collected by using the mitochondria collection needle. The protective solution used in the experiments is NaCl solution with the osmolarity of 42.8 mOsm/L.

The extraction efficiency of the mitochondria is obtained by a cell image counter. If the cells are damaged, the mitochondria will be released from the cells. The cell image counter counts the number of the total cells and the damaged cells. The ratio of the damaged cells to the total cells is defined as the extraction efficiency.

The function of the mitochondria is determined by measuring the membrane potential. Tetramethylrhodamine ethyl ester (TMRE) is a fluorescent dye with positive charge. TMRE can gather on the healthy mitochondria so that TMRE can be used to label the healthy mitochondria. When the mitochondria are less active or depolarized, the membrane potential decreases, and TMRE cannot be retained on the mitochondria. Carbonylcyanide 4-(trifluoromethoxy) phenylhydrazone (FCCP) is an ionophore that can cross the inner membranes of the mitochondria. FCCP combines protons to disrupt the synthesis of ATP and cause changes in the membrane potential. FCCP is able to eliminate the membrane potential and thus often used to deactivate or depolarize the mitochondria. The changes in the membrane potential are analyzed by fluorescent analysis through the treatment of TMRE and FCCP to determine the function of the mitochondria. The cells treated with TMRE are detected by a flow cytometer, and the ratio of the functional mitochondria to the total particles is analyzed based on TMRE analysis.

The mass of the mitochondria is measured by Pierce™ BCA Protein Assay Kit. For the operation procedure, please refer to the guideline of this kit. The bovine serum albumin (BSA) is used as the standard in this measurement, and the stock solution of BSA is 2 μg/mL. The reagent A (colorless) and the reagent B (blue) in Pierce™ BCA Protein Assay Kit are prepared in a ratio of 50:1 as the working reagent. The preparation of the standard solution is shown in Table 1. Samples are measured at 562 nm by the spectrophotometer, and the mitochondria mass in the samples is calculated based on the standard curve. In Table 1, the blank is used to correct the background, and the samples are the supernatant comprising mitochondria after extraction.

The purity of the mitochondria herein represents the percentage of the functional mitochondria to the total particles in the supernatant and is analyzed by fluorescent analysis through the treatment of only TMRE.

The content of the mitochondria herein represents the percentage of the all mitochondria to the total particles in the supernatant and is analyzed by 10-N-Nonyl acridine orange (NAO) staining. NAO is an acridine orange derivative and is generally used as a fluorescent marker of the inner mitochondrial membrane so that NAO can stain functional and non-functional mitochondria for analyzing the content of mitochondria.

TABLE 1

| BSA (μg/mL) | |
|---|---|
| — | blank |
| — | 198 μL working reagent + 2 μL sample |
| 0 | 200 μL working reagent |
| 40 | 198 μL working reagent + 2 μL BSA |
| 80 | 196 μL working reagent + 4 μL BSA |
| 120 | 194 μL working reagent + 6 μL BSA |
| 160 | 192 μL working reagent + 8 μL BSA |
| 200 | 190 μL working reagent + 10 μL BSA |

Experiment 1

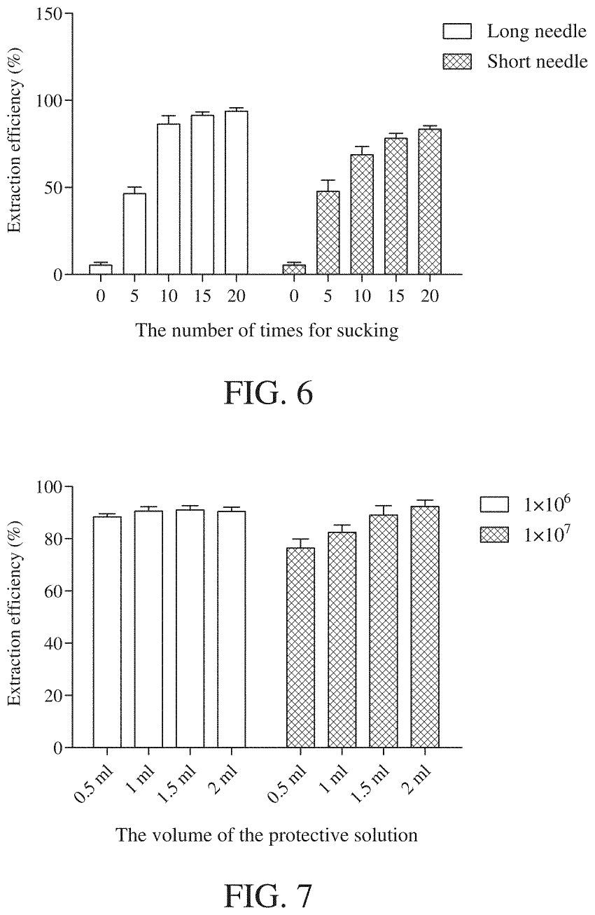

In Experiment 1, the extraction efficiency for different numbers of times for sucking the mixed solution back and forth and different lengths of the sucking needle is studied. This experiment is conducted by the method using the kit according to the second embodiment of the present disclosure. In this experiment, each group contains 1×10⁶ cells of the peripheral blood mononuclear cells, the protective solution is 1 mL, and the time for standing is 5 min. In addition, the long needle is a needle of 23 G (0.337 mm of the inner diameter) and 70 mm, the short needle is a needle of 23 G (0.337 mm of the inner diameter) and 15 mm, and the number of times for sucking back and forth is 0, 5, 10, 15, or 20 times. The result is shown in FIG. 6, and FIG. 6 shows the extraction efficiency for different numbers of times for sucking back and forth and different lengths of the needle.

The cells in the mixed solution are rubbed and collided with the inner wall of the needle to damage the cell membrane, thereby releasing the mitochondria. Thus, the longer needle and the more times for sucking back and forth cause the longer path that the cells are subjected to friction and the more friction and collision, and this facilitates the increase of the efficiency for damaging the cell membrane. Therefore, as shown in FIG. 6, fewer times for sucking back and forth causes the lower extraction efficiency, the increasing times for sucking back and forth increases the extraction efficiency, but the excessive times for sucking back and forth do not have more help for the extraction efficiency. In addition, under the same times for sucking back and forth, the extraction efficiency of the long needle is superior to that of the short needle. According to the result of this experiment, even if the short needle is used and the times for sucking back and forth is 5 times, it still obtains approximately 50% of the extraction efficiency. When the long needle is used and the times for sucking back and forth is 15 times, it obtains close to 100% of the extraction efficiency.

Experiment 2

In Experiment 2, the cell numbers for per milliliter of the protective solution is studied, and the mitochondria are isolated from peripheral blood mononuclear cells. This experiment is conducted by the method using the kit according to the second embodiment of the present disclosure. In this experiment, the protective solution is NaCl solution with the osmolarity of 42.8 mOsm/L, the sucking needle is a needle of 23 G (0.337 mm of the inner diameter) and 70 mm in length, the number of times for sucking back and forth of the protective solution along with the peripheral blood mononuclear cells therein is 15 times, and the time for standing after sucking is 5 mins. For the first part, each group contains 1×10⁶ or 1×10⁷ cells of the peripheral blood mononuclear cells, and the protective solution is 0.5, 1, 1.5, or 2 mL. For the second part, 12 mL of the protective solution is used for 3×10⁸ or 3×10⁹ cells of the peripheral blood mononuclear cells, that is, 2.5×10⁷ or 2.5×10⁸ cells/mL for each group. The result of the first part is shown in FIG. 7 and Table 2, FIG. 7 shows the extraction efficiency for different numbers of the cells and different volumes of the protective solution. The result of the second part is shown in Table 2.

As shown in FIG. 7 and Table 2, when the cell number is 133 10⁶ cells, 1 mL of the protective solution has excellent extraction efficiency of 80% or more. When the cell number is 1×10⁷ cells, 2 mL of the protective solution has excellent extraction efficiency of 80% or more. When the cell number is 1×10⁶ to 2.5×10⁸cells/mL, the protective solution has good extraction efficiency of 70% or more. According to the result of this experiment, each milliliter of the protective solution that is NaCl solution with the osmolarity of 42.8 mOsm/L may be used for treating approximately 2.5×10⁸ or less cells, preferably 1×10⁶ to 2.5×10⁸ cells. Preferably, each milliliter of the protective solution that is NaCl solution with the osmolarity of 42.8 mOsm/L may be used for treating approximately 2.5×10⁸ or less cells of peripheral blood mononuclear cells, preferably 1×10⁶ to 2.5×10⁸ cells of peripheral blood mononuclear cells.

TABLE 2

| | Cell | | | |
|---|---|---|---|---|
| | Peripheral blood mononuclear cell | | | |
| | Cell number (cells/mL) | | | |
| | 1 × 10⁶ | 5 × 10⁶ | 2.5 × 10⁷ | 2.5 × 10⁸ |
| Extraction | 90.83 ± 3.03 | 92.5 ± 7.23 | 95.07 ± 3.77 | 71.17 ± 6.14 |
| efficiency (%) | | | | |
| Purity (%) | 15.7 ± 4.97 | 36.15 ± 7.22 | 49.41 ± 3.77 | 47.3 ± 6.66 |
| Content (%) | 69.58 ± 9.61 | 75.95 ± 6.19 | 76.55 ± 7.11 | 72.53 ± 5.95 |

Experiment 3

In Experiment 3, the extraction efficiency for different times for standing is studied.

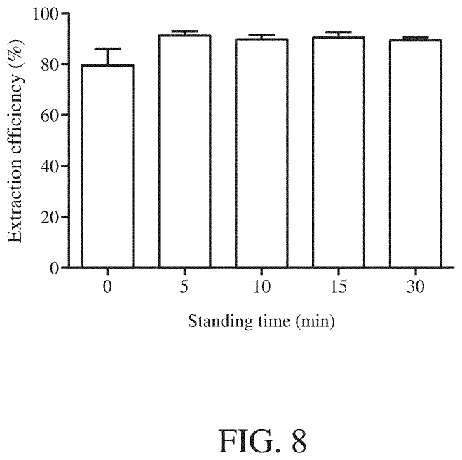

This experiment is conducted by the method using the kit according to the second embodiment of the present disclosure. In this experiment, each group contains 1×10⁶ cells of the peripheral blood mononuclear cells, the sucking needle is a needle of 23 G (0.337 mm of the inner diameter) and 70 mm, the number of times for sucking back and forth is 15 times, and the protective solution is 1 mL. In addition, the time for standing is 0, 5, 10, 15, or 30 min. The result is shown in FIG. 8, FIG. 8 shows the extraction efficiency for different times for standing.

Standing of the mixed solution allows the hypotonic protective solution has enough time to diffuse to facilitate the further destruction of the damaged cell membranes, thereby increasing the efficiency for destroying the cell membrane. As shown in FIG. 8, after sucking the mixed solution back and forth, the mixed solution that stands for minutes has a better extraction efficiency than the mixed solution without standing. According to the result of this experiment, when the mixed solution stands for at least 5 min, it obtains above 80% of the extraction efficiency, and there is no significant change if being stood for longer time.

Experiment 4

In Experiment 4, the mitochondria are isolated from the peripheral blood mononuclear cells. In this experiment, the number of the peripheral blood mononuclear cells in the blood (about 8 mL of the peripheral blood) is approximately 2.5×10⁶, the protective solution is 1 mL of NaCl solution with the osmolarity of 42.8 mOsm/L, the sucking needle is a needle of 23 G (0.337 mm of the inner diameter) and 70 mm, the number of times for sucking back and forth is 15 times, and the time for standing is 5 min. The result is shown in Table 3. There are 8.30 μg of the mitochondria are isolated from 2.5×10⁶ cells of the peripheral blood mononuclear cells, and the percentage of the functional mitochondria to the total particles in the supernatant, represented as purity in Table 3, is 48.55%.

Experiment 5

In Experiment 5, the mitochondria are isolated from the adipose-derived stem cells. In this experiment, the adipose-derived stem cells are approximately 5×10⁶ cells, the protective solution is 1 mL of NaCl solution with the osmolarity of 42.8 mOsm/L, the sucking needle is a needle of 23 G (0.337 mm of the inner diameter) and 70 mm, the number of times for sucking back and forth is 15 times, and the time for standing is 5 min. The result is shown in Table 3. There are 10.97 μg of the mitochondria are isolated from 5×10⁶ cells of the adipose-derived stem cells, and the percentage of the functional mitochondria to the total particles in the supernatant, represented as purity in Table 3, is 39.68%.

TABLE 3

| | Experiment 4 | Experiment 5 |
|---|---|---|
| Cell | peripheral blood | adipose-derived |
| | mononuclear cell | stem cell |
| Cell number | 2.5 × 10⁶ | 5 × 10⁶ |
| Mitochondria mass (μg) | 8.30 | 10.97 |
| Purity (%) | 48.55 | 39.68 |

The embodiments according to the present disclosure provide a kit and a method for isolating mitochondria. The cell membranes of the cells may be destroyed by the sucking needle and the protective solution in the kit through the friction of the cells in the sucking needle with the help of the protective solution, and this way of destroying the cell membranes may not damage the mitochondria in the cells. Therefore, the mitochondria may be isolated from cells with high efficiency in a simple and convenient way, and the isolated mitochondria may have excellent function and activity.

The protective solution in the kit for isolating mitochondria according to the present disclosure will be further described below.

The protective solution is a solution used for isolating mitochondria from cells and protecting the isolated mitochondria. The osmolarity of the protective solution may be greater than 0 and less than or equal to 220 mOsm/L. In some embodiments, the osmolarity of the protective solution may be 42.8 mOsm/L to 220 mOsm/L. In some embodiments, the osmolarity of the protective solution may be 42.8 mOsm/L to 113 mOsm/L. In some embodiments, the protective solution may comprise NaCl, glucose, NaH₂PO₄, or mannitol. In some embodiments, the protective solution may comprise NaCl and glucose, and the weight ratio of NaCl to glucose may be 1:0.06 to 1:2560. In some embodiments, the protective solution may comprise NaCl and NaH₂PO₄, and the weight ratio of NaCl to NaH₂PO₄ may be 1:0.015 to 1:133. In some embodiments, the protective solution may comprise glucose and NaH₂PO₄, and the weight ratio of glucose to NaH₂PO₄ may be 1:0.0007 to 1:22. In some embodiments, the protective solution may only comprise NaCl and glucose without other solutes. In some embodiments, the protective solution may only comprise NaCl and NaH₂PO₄ without other solutes. In some embodiments, the protective solution may only comprise glucose and NaH₂PO₄ without other solutes.

The composition and the osmolarity of the examples (Ex.) of the protective solution according to the present disclosure and the comparative examples (Com.) of the extraction solution are shown in Table 4 and 5.

TABLE 4

| | | Composition | | | |
|---|---|---|---|---|---|
| | | NaCl | Glucose | NaH₂PO₄ | Mannitol |
| Osmolarity | 42.8 | Ex. 1 | Ex. 4 | Ex. 7 | Ex. 10 |
| (mOsm/L) | 113 | Ex. 2 | Ex. 5 | Ex. 8 | Ex. 11 |
| | 220 | Ex. 3 | Ex. 6 | Ex. 9 | Ex. 12 |
| | 520 | Com. 1 | Com. 3 | Com. 5 | Com. 7 |
| | 1025 | Com. 2 | Com. 4 | Com. 6 | Com. 8 |

TABLE 5

| | Composition | Osmolarity (mOsm/L) |
|---|---|---|
| Ex. 13 | NaCl + Glucose | 42.8 |
| Ex. 14 | NaCl + NaH₂PO₄ | 42.8 |
| Ex. 15 | Glucose + NaH₂PO₄ | 42.8 |

Experiments 6 to 8 demonstrate isolating mitochondria by the protective solution according to the present disclosure. The mitochondria are isolated by using the examples (Ex.) of the protective solution according to the present disclosure and the comparative examples (Com.) of the extraction solution, according to the method and the kit of the second embodiment of the present disclosure, and the function and the extraction efficiency of the isolated mitochondria are analyzed.

Specifically, firstly, the cells and the protective solution are mixed to form the mixed solution. In detail, a certain number of the cells (1×10⁶ cells) are collected. The cells are the peripheral blood mononuclear cells or the adipose-derived stem cells, but the present disclosure is not limited thereto. The cells may be any cells that have mitochondria. Then, the cells and 1 mL of the protective solution of the examples or 1 mL of the extraction solution of the comparative examples are mixed to form the mixed solution.

Subsequently, the cells in the mixed solution are rubbed to damage the cell membranes of the cells to facilitate the entry of the protective solution into the cells and the destruction of the cell membranes of the cells. In detail, during mixing the cells and the protective solution, the cell membranes of the cells is damaged due to friction. The damaged cell membranes facilitate the entry of the protective solution into the cells, thereby further destroying the cell membrane. Here, a long needle of 23 G and 70 mm is used to suck the mixed solution back and forth to damage the cells, but the present disclosure is not limited thereto. When using the protective solution according to the present disclosure, a needle of different sizes may be used corresponding to the experimental equipment, or machines such a grinder may be used, thereby damaging the cell membranes to facilitate the entry of the protective solution into the cells so as to further destroy the cell membrane.

Subsequently, the mixed solution is centrifuged to be stratified. In detail, the mixed solution stands and then centrifuged, and the mixed solution is stratified to form the supernatant comprising the mitochondria and the pellet comprising cell debris.

Subsequently, the supernatant comprising the mitochondria is collected. In detail, the supernatant comprising the mitochondria may be collected by using needle or pipette or by pouring.

Finally, the supernatant comprising the mitochondria is analyzed for the extraction efficiency and the function of the mitochondria.

Experiment 6

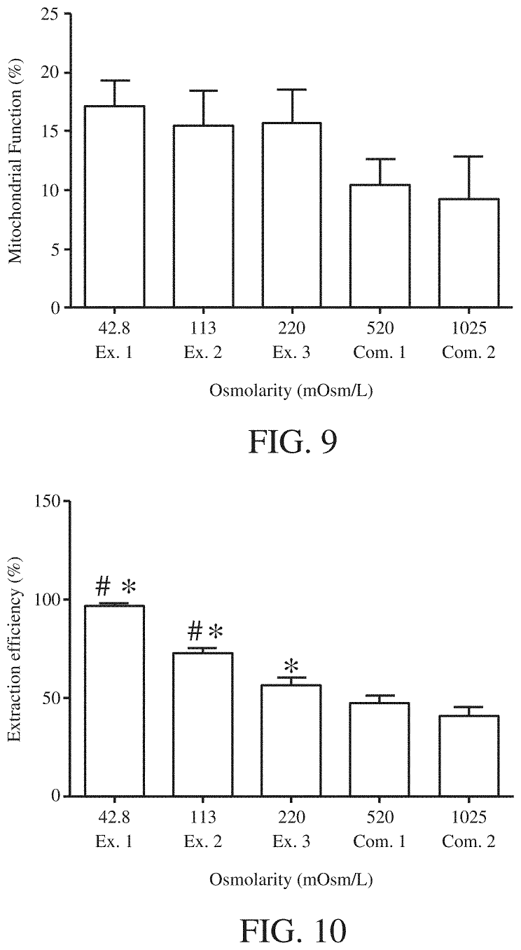

In Experiment 6, the mitochondria are isolated from the peripheral blood mononuclear cells by using the protective solution comprising NaCl with different osmolarities, and the extraction efficiency and the function of the mitochondria are analyzed. Please refer to FIGS. 9 and 10, wherein FIG. 9 shows the function of mitochondria isolated from the peripheral blood mononuclear cells by the protective solution comprising NaCl with different osmolarities, and FIG. 10 shows the extraction efficiency of mitochondria isolated from the peripheral blood mononuclear cells by the protective solution comprising NaCl with different osmolarities. In FIG. 10, “#” indicates a significant difference (P<0.05) compared with the comparative example of 520 mOsm/L, “*” indicates a significant difference (P<0.05) compared with the comparative example of 1025 mOsm/L.

From FIG. 9 and Table 6, it can be seen that by using the protective solution comprising NaCl with the osmolarity greater than 0 and less than or equal to 220 mOsm/L to isolate mitochondria, the isolated mitochondria have excellent function, and the function of the mitochondria is greater than 10%. From FIG. 10 and Table 6, it can be seen that by using the protective solution comprising NaCl with the osmolarity greater than 0 and less than or equal to 220 mOsm/L to isolate mitochondria, excellent extraction efficiency is obtained, and the extraction efficiency is greater than 50%. Taking together, by using the protective solution comprising NaCl with the osmolarity greater than 0 and less than or equal to 220 mOsm/L to isolate mitochondria from the peripheral blood mononuclear cells, the mitochondria maintain their function (greater than 10%) while obtaining the excellent extraction efficiency (greater than 50%).

TABLE 6

| | Osmolarity | | Extraction |
|---|---|---|---|
| | (mOsm/L) | Function (%) | efficiency (%) |
| Ex. 1 | 42.8 | 17.2 ± 3.8 | 97.0 ± 2.0 |
| Ex. 2 | 113 | 15.4 ± 5.2 | 72.7 ± 4.2 |
| Ex. 3 | 220 | 15.7 ± 5.0 | 56.7 ± 6.5 |
| Com. 1 | 520 | 10.4 ± 3.8 | 47.6 ± 5.9 |
| Com. 2 | 1025 | 9.2 ± 5.1 | 40.6 ± 7.9 |

Cell: peripheral blood mononuclear cell

Experiment 7

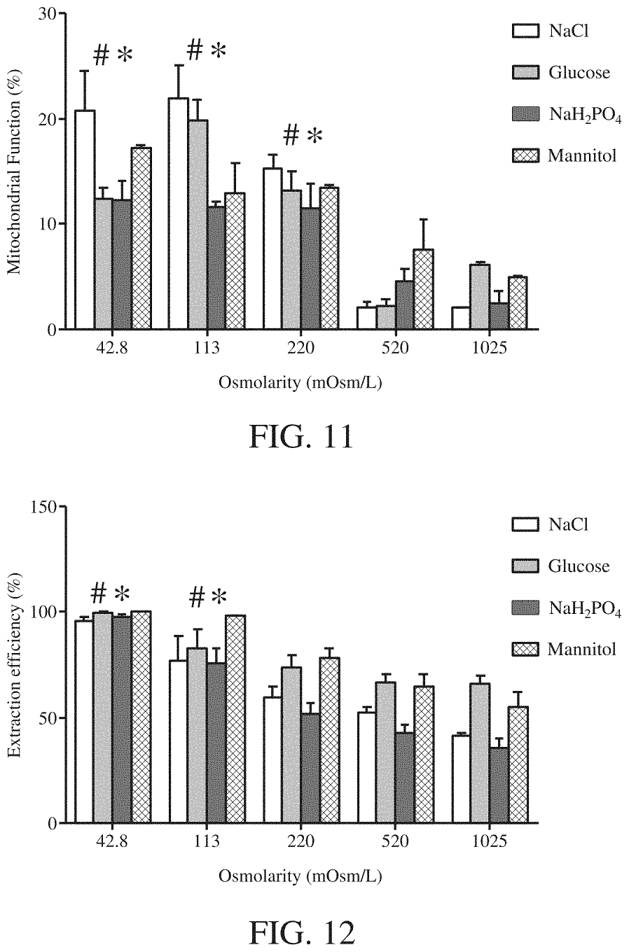

In Experiment 7, the mitochondria are isolated from the adipose-derived stem cells by using the protective solution comprising NaCl, glucose, NaH₂PO₄, or mannitol with different osmolarities, and the extraction efficiency and the function of the mitochondria are analyzed. Please refer to FIGS. 11 and 12, wherein FIG. 11 shows the function of mitochondria isolated from the adipose-derived stem cells by the protective solution comprising NaCl, glucose, NaH₂PO₄, or mannitol with different osmolarities, and FIG. 12 shows the extraction efficiency of mitochondria isolated from the adipose-derived stem cells by the protective solution comprising NaCl, glucose, NaH₂PO₄, or mannitol with different osmolarities. In FIGS. 11 and 12, “#” indicates a significant difference (P<0.05) compared with the comparative example of 520 mOsm/L, “*” indicates a significant difference (P<0.05) compared with the comparative example of 1025 mOsm/L.

From FIG. 11 and Table 7, it can be seen that by using the protective solution comprising NaCl, glucose, NaH₂PO₄, or mannitol with the osmolarity greater than 0 and less than or equal to 220 mOsm/L to isolate mitochondria, the isolated mitochondria have excellent function, and the function of the mitochondria is greater than 10%. From FIG. 12 and Table 7, it can be seen that by using the protective solution comprising NaCl, glucose, NaH₂PO₄, or mannitol with the osmolarity greater than 0 and less than or equal to 220 mOsm/L to isolate mitochondria, excellent extraction efficiency is obtained, and the extraction efficiency is greater than 50%. Taking together, by using the protective solution comprising NaCl, glucose, NaH₂PO₄, or mannitol with the osmolarity greater than 0 and less than or equal to 220 mOsm/L to isolate mitochondria from the adipose-derived stem cells, the mitochondria maintain their function (greater than 10%) while obtaining the excellent extraction efficiency (greater than 50%).

TABLE 7

| | | |
|---|---|---|
| Osmolarity | | Extraction |
| (mOsm/L) | Function (%) | efficiency (%) |

NaCl

| | | | |
|---|---|---|---|
| Ex. 1 | 42.8 | 20.7 ± 6.5 | 95.9 ± 3.6 |
| Ex. 2 | 113 | 21.9 ± 5.4 | 76.9 ± 20.1 |
| Ex. 3 | 220 | 14.6 ± 1.8 | 59.4 ± 9.4 |
| Com. 1 | 520 | 2.0 ± 1.0 | 52.6 ± 3.8 |
| Com. 2 | 1025 | 2.0 ± 0.2 | 41.5 ± 2.4 |

Glucose

| | | | |
|---|---|---|---|
| Ex. 4 | 42.8 | 12.4 ± 1.8 | 99.5 ± 0.9 |
| Ex. 5 | 113 | 19.8 ± 3.4 | 82.9 ± 14.9 |
| Ex. 6 | 220 | 13.1 ± 2.6 | 73.9 ± 9.8 |
| Com. 3 | 520 | 2.2 ± 1.1 | 66.4 ± 7.0 |
| Com. 4 | 1025 | 6.1 ± 0.5 | 65.8 ± 6.9 |

NaH₂PO₄

| | | | |
|---|---|---|---|
| Ex. 7 | 42.8 | 12.3 ± 2.9 | 97.7 ± 2.1 |
| Ex. 8 | 113 | 11.6 ± 0.9 | 75.5 ± 12.7 |
| Ex. 9 | 220 | 11.5 ± 4.0 | 51.6 ± 9.7 |
| Com. 5 | 520 | 4.5 ± 2.0 | 42.7 ± 6.7 |
| Com. 6 | 1025 | 2.4 ± 2.1 | 35.9 ± 7.8 |

Mannitol

| | | | |
|---|---|---|---|
| Ex. 10 | 42.8 | 17.2 ± 0.5 | 100 ± 0 |
| Ex. 11 | 113 | 13.0 ± 4.8 | 98.2 ± 0.4 |
| Ex. 12 | 220 | 13.4 ± 0.5 | 78.3 ± 7.9 |
| Com. 7 | 520 | 7.5 ± 5.1 | 64.8 ± 9.5 |
| Com. 8 | 1025 | 5.0 ± 0.2 | 55 ± 12.0 |

Cell: adipose-derived stem cell

Experiment 8

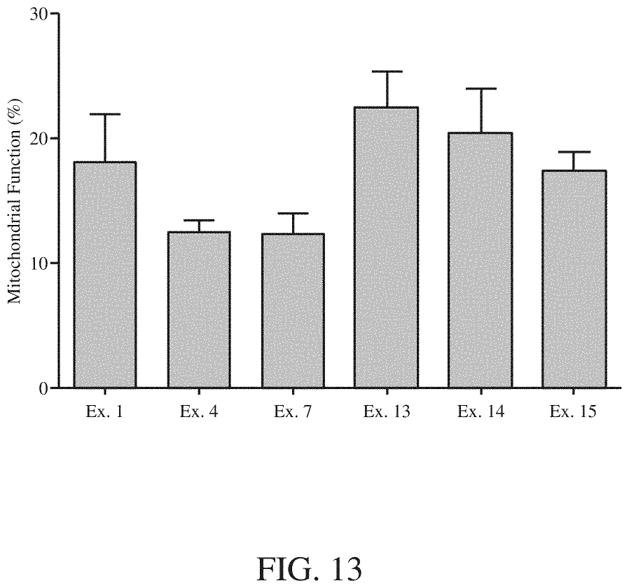

In Experiment 8, the mitochondria are isolated from the adipose-derived stem cells by using the protective solution comprising different compositions with an osmolarity of 42.8 mOsm/L, and the function of the mitochondria is analyzed. Please refer to FIG. 13 and Table 8, wherein FIG. 13 shows the function of mitochondria isolated from the adipose-derived stem cells by the protective solution comprising different compositions with an osmolarity of 42.8 mOsm/L.

From FIG. 13 and Table 8, it can be seen that under the osmolarity of 42.8 mOsm/L, by using the protective solution comprising single composition (Ex. 1, NaCl; Ex. 4, glucose; Ex. 7, NaH₂PO₄) to isolate mitochondria from the adipose-derived stem cells, the isolated mitochondria have excellent function, and the function of the mitochondria is greater than 10%. In addition, under the osmolarity of 42.8 mOsm/L, by using the protective solution comprising two of NaCl, glucose, and NaH₂PO₄ (Ex. 13, NaCl and glucose; Ex. 14, NaCl and NaH₂PO₄; Ex. 15, Glucose+NaH₂PO₄) to isolate mitochondria from the adipose-derived stem cells, the isolated mitochondria have more excellent function, and the function of the mitochondria is greater than 15%.

TABLE 8

| | Composition | Function (%) |
|---|---|---|
| Ex. 1 | NaCl | 18.0 ± 7.8 |
| Ex. 4 | Glucose | 12.4 ± 1.8 |
| Ex. 7 | NaH₂PO₄ | 12.3 ± 2.9 |
| Ex. 13 | NaCl + Glucose | 22.5 ± 5.8 |
| Ex. 14 | NaCl + NaH₂PO₄ | 20.4 ± 6.1 |
| Ex. 15 | Glucose + NaH₂PO₄ | 17.3 ± 2.7 |

Cell: adipose-derived stem cell

Experiment 9

In Experiment 9, the cell numbers for per milliliter of the protective solution is studied, and the mitochondria are isolated from adipose-derived stem cells. This experiment is conducted by the method using the kit according to the second embodiment of the present disclosure. In this experiment, the protective solution is NaCl solution with the osmolarity of 42.8 mOsm/L. The sucking needle is a needle of 23 G (0.337 mm of the inner diameter) and 70 mm in length, the number of times for sucking back and forth of the protective solution along with the adipose-derived stem cells therein is 15 times, and the time for standing after sucking is 5 mins. 12 mL of the protective solution is used for 3×10⁸ or 3×10⁹ cells of the adipose-derived stem cells, that is, 2.5×10⁷ or 2.5×10⁸ cells/mL for each group. The result is shown in Table 9.

From Table 9, it can be seen that when the protective solution is used at 2.5×10⁷ or 2.5×10⁸ cells/mL, a good extraction efficiency which is greater than 65% is obtained. The percentage of the functional mitochondria to the total particles in the supernatant, represented as purity in Table 9, is 50.22% and 52.6% for each group. The percentage of all mitochondria to the total particles in the supernatant, represented as content in Table 9, is 78.91% and 73.8% for each group. Further, when the protective solution is used at 2.5×10⁷ cells/mL, an excellent extraction efficiency which is greater than 90% is obtained.

From Experiment 9, it can be seen that each milliliter of the protective solution may be used for treating approximately 2.5×10⁸ or less cells, preferably 2.5×10⁷ to 2.5×10⁸ cells. Preferably, each milliliter of the protective solution that is NaCl solution with the osmolarity of 42.8 mOsm/L may be used for treating approximately 2.5×10⁸ or less cells of adipose-derived stem cells, preferably 2.5×10⁷ to 2.5×10⁸ cells for adipose-derived stem cells.

TABLE 9

Cell
Adipose-derived stem cell

| Cell number (cells/mL) | 2.5 × 10⁷ | 2.5 × 10⁸ |
|---|---|---|
| Extraction efficiency (%) | 93.11 ± 2.10 | 67.2 ± 3.80 |
| Purity (%) | 50.22 ± 9.80 | 52.6 ± 5.39 |
| Content (%) | 78.91 ± 7.47 | 73.8 ± 6.09 |

According to the above results, using a hypotonic solution has better extraction efficiency than using a hypertonic solution. In addition, the above experiments demonstrate that by using the protective solution of the osmolarity greater than 0 and less than or equal to 220 mOsm/L to isolate mitochondria, the mitochondria maintain their function while obtaining the excellent extraction efficiency. Further, the above experiments demonstrate that by using the protective solution comprising two of NaCl, glucose, and NaH₂PO₄ to isolate mitochondria, the isolated mitochondria have more excellent function. In different experiments, the differences in numerical values may be derived from the experimental error in each batch of experiments, and these differences in numerical values are in the acceptable range in the art.

The embodiments according to the present disclosure provide a protective solution for isolating mitochondria from cells and protecting the isolated mitochondria. When damaging the cell membrane, for example, when using a long needle to suck back and forth to damage the cell membranes due to friction, the osmolarity of the protective solution is greater than 0 and less than or equal to 220 mOsm/L so that the cell membranes of the cells may be destroyed with the help of the hypotonicity of the protective solution to release the mitochondria, and this way of destroying the cell membranes may not damage the mitochondria in the cells. Therefore, the mitochondria may be isolated from cells with high efficiency in a simple and convenient way, and the isolated mitochondria may have excellent function and activity.

Though the embodiment according to the present disclosure is described above, the present disclosure is not limited thereto. Without departing from the spirit and scope of the present disclosure, any skilled person in the field can do some appropriate change in the shapes, structures, characteristics and spirits. The extent of patent protection subject to the claim in the specification.

What is claimed is:

1. A kit for isolating mitochondria, comprising:
an extraction tube for containing cells;
a protective solution for forming a mixed solution with the cells in the extraction tube; and
a sucking needle for sucking the mixed solution back and forth.

2. The kit according to claim 1, wherein a length of the sucking needle is 15 mm or more.

3. The kit according to claim 1, wherein an inner diameter of the sucking needle is 0.318 mm to 0.356 mm.

4. A method for isolating mitochondria, comprising:
mixing cells and a protective solution to form a mixed solution, with a osmolarity of the protective solution greater than 0 and less than or equal to 220 mOsm/L;
rubbing the cells in the mixed solution to damage cell membranes of the cells to facilitate an entry of the protective solution into the cells and destroy the cell membranes of the cells;
centrifuging the mixed solution; and
collecting supernatant comprising mitochondria obtained after centrifuging.

5. The method according to claim 4, wherein in the step of rubbing the cells, a sucking needle is used for sucking the mixed solution back and forth for rubbing the cells.

6. The method according to claim 4, before the step of mixing the cells and the protective solution, further comprising:
injecting a solution comprising the cells to an extraction tube by using a cell collection needle;
centrifuging the solution; and
removing the supernatant without the cells and retaining the cells in the extraction tube.

7. The method according to claim 4, wherein in the step of collecting the supernatant comprising mitochondria, a mitochondria collection needle is used.

8. The method according to claim 4, wherein in the step of mixing the cells and the protective solution, a ratio of a number of the cells to a volume of the protective solution is 2.5×10⁸ cells or less per milliliter.

9. The method according to claim 5, wherein when a length of the sucking needle is 70 mm, a number of times for sucking the mixed solution back and forth is at least five.

10. The method according to claim 5, after the step of sucking the mixed solution back and forth by using the sucking needle, further comprising leaving the mixed solution to stand for an equilibrium time, the equilibrium time is at least five minutes.

11. A protective solution for isolating and protecting mitochondria from cells, wherein a osmolarity of the protective solution is greater than 0 and less than or equal to 220 mOsm/L.

12. The protective solution according to claim 11, wherein the osmolarity of the protective solution is 42.8 mOsm/L to 113 mOsm/L.

13. The protective solution according to claim 11, comprising sodium chloride.

14. The protective solution according to claim 11, comprising glucose.

15. The protective solution according to claim 11, comprising sodium dihydrogen phosphate.

16. The protective solution according to claim 11, comprising mannitol.

17. The protective solution according to claim 11, comprising sodium chloride and glucose, wherein a weight ratio of sodium chloride to glucose is 1:0.06 to 1:2560.

18. The protective solution according to claim 11, comprising sodium chloride and sodium dihydrogen phosphate, wherein a weight ratio of sodium chloride to sodium dihydrogen phosphate is 1:0.015 to 1:133.

19. The protective solution according to claim 11, comprising glucose and sodium dihydrogen phosphate, wherein a weight ratio of glucose to sodium dihydrogen phosphate is 1:0.0007 to 1:22.

20. A use of a protective solution according to claim 11 in isolating mitochondria from cells and maintaining the activity of mitochondria, wherein the protective solution comprises sodium chloride, glucose, sodium dihydrogen phosphate, or mannitol.
